(19) United States
(12) Patent Application Publication
(10) Pub. No.: US 2026/0191472 A1
(43) Pub. Date: Jul. 9, 2026

(54) SENSOR-CARRYING CATHETER SHAFT SEGMENT AND METHOD OF ITS MANUFACTURE

(71) Applicant: St. Jude Medical, Cardiology Division, Inc., St. Paul, MN (US)

(72) Inventors: Mitchell Armstrong, Eden Prairie, MN (US); Greg Hamilton, St. Paul Park, MN (US); Alissa Johnson, New Hope, MN (US); Emma Gantzer, Minneapolis, MN (US); Jack Hines, Chaska, MN (US); Jack Zofkie, Minneapolis, MN (US); Hannah Mitchell, Minneapolis, MN (US); Brian Jones, St. Paul, MN (US); Zach Helgeson, Richfield, MN (US); Katie Batman, Saint Louis Park, MN (US); Arun Kumar, Maple Grove, MN (US); Jason Buysman, Minnetonka, MN (US)

(21) Appl. No.: 19/130,814

(22) PCT Filed: Nov. 17, 2023
(86) PCT No.: PCT/US2023/080308

§ 371 Date: May 16, 2025

Related U.S. Application Data
(60) Provisional application No. 63/384,282, filed on Nov. 18, 2022.
(60) Provisional application No. 63/497,499, filed on Apr. 21, 2023.

(51) Int. Cl. A61B 5/00; A61B 5/25; A61M 25/00

(52) U.S. Cl. CPC A61B 5/6852; A61B 5/25; A61B 2562/04; A61B 2562/125; A61M 25/0009

(57) ABSTRACT

A medical device manufacturing process can include inserting a hollow shaft having a longitudinally-extending slit into the central lumen of a tubular device body, leaving its distal end protruding beyond the device body. The shaft includes an inner layer of a first material and an outer layer of a second material; the second material melts at a lower temperature than the first material. A sensor stack (14), including alternately-disposed spacing elements (14) and sensors (28), is formed around the protruding segment of the shaft. The spacing elements include an inner layer (34a) of a third material and an outer layer (34b) of a fourth material; the third material melts at a lower temperature than the fourth material. The assembly bonded by heating it above the melting temperatures of the second and third materials but below the those of the first and fourth materials.

### CROSS-REFERENCE TO RELATED APPLICATIONS

This application claims the benefit of U.S. provisional application nos. 63/384,282, filed 18 Nov. 2022 (“the ‘282 provisional”) and 63/497,499, filed 21 Apr. 2023 (“the ‘499 provisional”). The ‘282 and ‘499 provisionals are hereby incorporated by reference as though fully set forth herein.

### BACKGROUND

The present disclosure relates generally to elongate medical devices, such as catheters. In particular, the instant disclosure relates to elongate medical devices carrying various sensors, for example in their distal segments.

Catheters are used for an ever-growing number of procedures. For example, catheters are used for diagnostic, therapeutic, and ablative procedures, to name just a few examples. In an electrophysiology (“EP”) procedure, for example, a catheter may be manipulated through the patient's vasculature and to an intended site for mapping and/or treatment, for example, a site within the patient's heart.

A catheter may carry one or more devices, sensors, or surgical instruments, such as electrodes, which may be used for ablation, diagnosis, and/or the like. In many extant catheters, these sensors are embedded into the catheter shaft by swaging, bonded to the catheter shaft during a reflow process, and/or secured to the catheter shaft using adhesives.

These processes have certain disadvantages, however. For instance, the sensors must be relatively uniform metal bands in order to be swaged onto the catheter shaft. This makes swaging unsuitable for non-metallic and/or asymmetric sensors. Similarly, reflow and adhesive bonding processes add complexity, and therefore cost, to the manufacturing process.

### BRIEF SUMMARY

Disclosed herein is a method of securing at least one sensor to a medical device, including the steps: providing a tubular medical device body defining a central lumen; inserting a hollow shaft into the central lumen, wherein the hollow shaft includes an inner layer of a first material; an outer layer of a second material, wherein a melting temperature of the second material is lower than a melting temperature of the first material; and a slit through the inner layer and the outer layer and extending from a proximal end of the hollow shaft to a distal end of the hollow shaft, wherein a distal segment of the hollow shaft protrudes beyond a distal end of the tubular medical device body; forming a sensor stack around the protruding distal segment of the hollow shaft and abutting the distal end of the tubular medical device body, the sensor stack including at least one annular spacing element comprising an inner layer of a third material and an outer layer of a fourth material, wherein a melting temperature of the third material is lower than a melting temperature of the fourth material; and at least one annular sensor; and reflow bonding the sensor stack and the tubular medical device body to the hollow shaft.

The method can optionally include inserting a mandrel into the hollow shaft prior to reflow bonding the sensor stack and the tubular medical device body to the hollow shaft. The mandrel can be removed from the hollow shaft after reflow bonding the sensor stack and the tubular medical device body to the hollow shaft.

The method can also optionally include placing a heat shrink tube around the sensor stack prior to reflow bonding the sensor stack and the tubular medical device body to the hollow shaft.

In certain aspects of the disclosure, the at least one annular spacing element includes a plurality of annular spacing elements alternately arranged with the at least one annular sensor. Likewise, it is contemplated that the at least one annular sensor can include a plurality of annular sensors alternately arranged with the plurality of annular spacing elements.

In some embodiments, the at least one annular spacing element abuts the distal end of the tubular medical device body, and can optionally be secured to the distal end of the tubular medical device body via an adhesive.

The at least one annular sensor can include at least one ring electrode, such as at least one composite ring sensor including a non-conductive annular body defining a plurality of cavities; and a plurality of conductive elements respectively disposed in the plurality of cavities.

The method can also include coupling at least one signal conductor respectively to the at least one annular sensor; and routing the at least one signal conductor through the slit of the hollow shaft and into the central lumen of the tubular medical device body.

In certain embodiments, the second material is the same as the third material.

The step of reflow bonding the sensor stack and the tubular medical device body to the hollow shaft can include heating the sensor stack and the tubular medical device to a temperature above the melting temperature of the second material and the melting temperature of the third material but below the melting temperature of the first material and the melting temperature of the fourth material.

Optionally, the method can further include a primary swaging step in which the at least one annular sensor is swaged onto the protruding distal segment of the hollow shaft prior to the reflow bonding step and a secondary swaging step in which the at least one annular sensor is swaged onto the protruding distal segment of the hollow shaft after the reflow bonding step. The secondary swaging step can reduce the diameter of the at least one annular sensor by 0.0001 inches.

Also disclosed herein is a medical device, including: a tubular medical device body defining a central lumen; a hollow shaft inserted within the central lumen, wherein the hollow shaft includes an inner layer of a first material; an outer layer of a second material, wherein a melting temperature of the second material is lower than a melting temperature of the first material; and a slit through the inner layer and the outer layer and extending from a proximal end of the hollow shaft to a distal end of the hollow shaft, wherein a distal segment of the hollow shaft protrudes beyond a distal end of the tubular medical device body; and a sensor stack around the protruding distal segment of the hollow shaft and abutting the distal end of the tubular medical device body, wherein the sensor stack includes at least one annular spacing element comprising an inner layer of a third material and an outer layer of a fourth material, wherein a melting temperature of the third material is lower than a melting temperature of the fourth material; and at least one annular sensor; and wherein the sensor stack and the tubular medical device body are reflow bonded to the hollow shaft.

In embodiments of the disclosure, the at least one annular spacing element includes a plurality of annular spacing elements alternately arranged with the at least one annular sensor. Similarly, in embodiments of the disclosure, the at least one annular sensor includes a plurality of annular sensors alternately arranged with the plurality of annular spacing elements.

The at least one annular spacing element can abut the distal end of the tubular medical device body and can be adhesively secured to the distal end of the tubular medical device body.

It is contemplated that the at least one annular sensor can include at least one ring electrode. For example, the at least one ring electrode can include at least one composite ring sensor including a non-conductive annular body defining a plurality of cavities; and a plurality of conductive elements respectively disposed in the plurality of cavities.

At least one signal conductor may be respectively coupled to the at least one annular sensor and routed through the slit of the hollow shaft and into the central lumen of the tubular medical device body.

The instant disclosure also provides a method of manufacturing a medical device, including: providing a tubular medical device body defining a central lumen; inserting a hollow shaft into the central lumen, wherein the hollow shaft includes an inner layer of a non-reflowable material, an outer layer of a reflowable material, and a slit through the inner layer and the outer layer and extending from a proximal end of the hollow shaft to a distal end of the hollow shaft, such that a distal segment of the hollow shaft protrudes beyond a distal end of the tubular medical device body; forming a sensor stack around the protruding distal segment of the hollow shaft and abutting the distal end of the tubular medical device body, wherein the sensor stack includes a plurality of annular spacing elements alternately arranged with a plurality of annular sensors, and wherein each annular spacing element includes an inner layer of a reflowable material and an outer layer of a non-reflowable material; inserting a mandrel into the hollow shaft; and reflow bonding the respective inner layers of the plurality of annular spacing elements to the outer layer of the hollow shaft.

The foregoing and other aspects, features, details, utilities, and advantages of the present invention will be apparent from reading the following description and claims, and from reviewing the accompanying drawings.

### BRIEF DESCRIPTION OF THE DRAWINGS

FIG. 1 is a perspective view of an exemplary catheter according to embodiments of the instant disclosure.

FIG. 2 is a close-up view of the distal region of the exemplary catheter shown in FIG. 1.

FIGS. 3-7 depict various stages of a catheter body assembly according to aspects of the instant disclosure prior to reflow processing.

FIG. 8 is a perspective view of a catheter body assembly according to aspects of the instant disclosure prior to reflow processing.

FIG. 9 depicts a catheter body assembly according to aspects of the instant disclosure after reflow processing.

FIG. 10A is a top view of a composite sensor element, such as may be used in accordance with certain teachings herein.

FIG. 10B is a side view of the composite sensor element shown in FIG. 10A.

FIGS. 11A through 11C illustrate primary and secondary swaging steps that may reinforce or enhance the connection between a sensor element and a underlying shaft, such as a catheter body.

While multiple embodiments are disclosed, still other embodiments of the present disclosure will become apparent to those skilled in the art from the following detailed description, which shows and describes illustrative embodiments. Accordingly, the drawings and detailed description are to be regarded as illustrative in nature and not restrictive.

### DETAILED DESCRIPTION

Aspects of the instant disclosure relate to mounting various sensors on elongate medical devices. For purposes of illustration, embodiments of the disclosure will be described in connection with mounting electrodes on an elongate electrophysiology catheter. It is contemplated, however, that the described features and methods may be incorporated into any number of catheters or similar medical devices (e.g., steerable diagnostic and therapeutic catheters, fixed curve catheters and introducers, and the like).

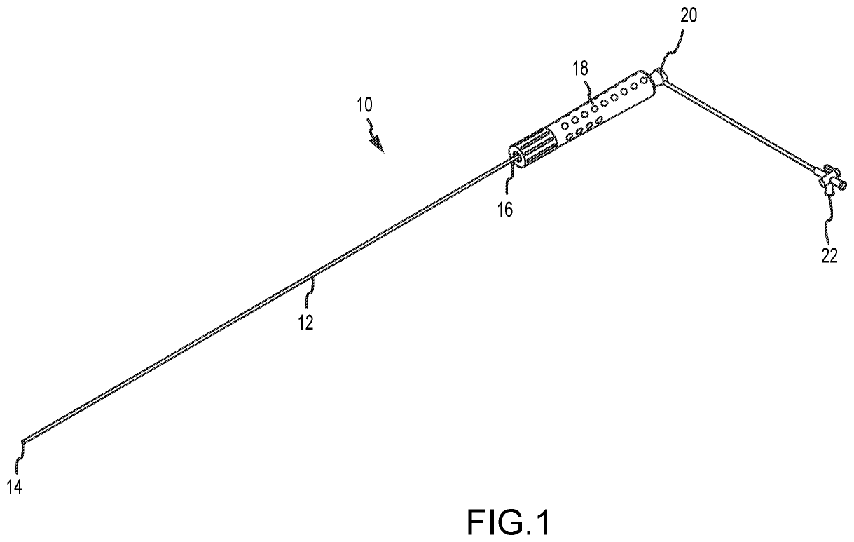

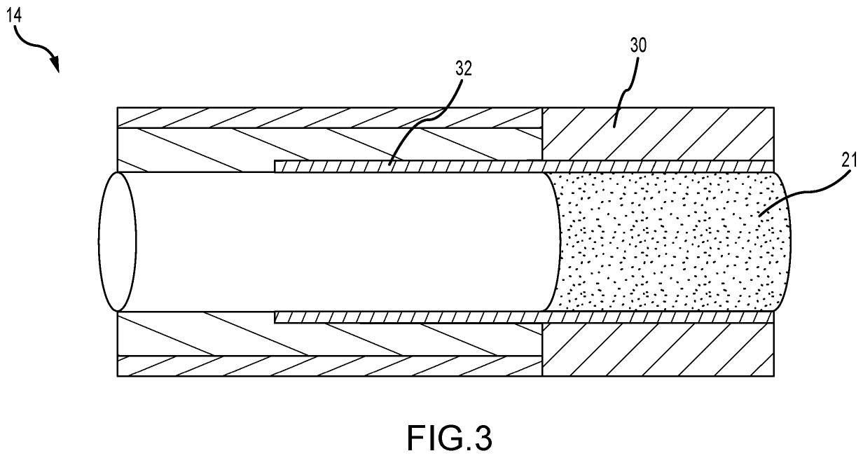

Referring now to the Figures, and in particular to FIG. 1, an electrophysiology catheter 10 includes an elongate catheter body 12 having a distal region 14 and a proximal end 16. A handle 18 may be coupled to proximal end 16 of catheter body 12 to control catheter 10 (e.g. to push, torque, and/or steer catheter 10). Catheter 10 may also include a hub 20 operably coupled to a central lumen 21 (not shown in FIG. 1, but visible in FIGS. 3-7 and 9) within catheter body 12. A valve 22 may be operably connected to hub 20. Of course, it is also contemplated that any known device for manipulation of catheter 10 may be coupled to proximal end 16 of body 12, including, without limitation, robotic manipulation devices and the like.

Various additional (and, in some instances, optional) aspects of the construction of catheter 10 will be familiar to those of ordinary skill in the art. For example, the person of ordinary skill in the art will recognize that catheter 10 can be made steerable, for example by incorporating one or more actuators into handle 18 that are coupled to one or more steering or pull wires that extend through body 12 and that terminate in one or more pull rings within distal region 14. Likewise, the ordinarily skilled artisan will appreciate that catheter 10 can be an irrigated catheter, such that it can also be coupled to a suitable supply of irrigation fluid and/or an irrigation pump (e.g., a peristaltic pump). As a further example, those of ordinary skill in the art will appreciate that catheter 10 can be equipped with force feedback capabilities.

Insofar as such features are not necessary to an understanding of the instant disclosure, they are neither illustrated in the drawings nor explained in detail herein. By way of example only, however, catheter 10 can incorporate various aspects and features of the following catheters, all from Abbott Laboratories (Abbott Park, IL): the Advisor™ HD Grid Mapping Catheter, Sensor Enabled™ (SE); the FlexAbility™ ablation catheter; the Safire™ BLU™ ablation catheter; the Therapy™ Cool Path™ irrigated ablation catheter; the Livewire™ TC ablation catheter; the TactiCath™ Contact Force Ablation Catheter, Sensor Enabled™ (SE); and the TactiFlex™ Ablation Catheter, Sensor Enabled™ (SE).

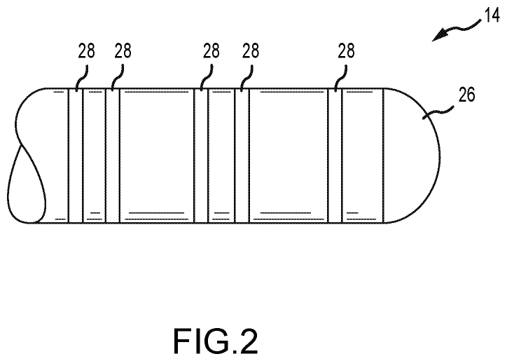

FIG. 2 is a close-up of distal region 14 of catheter 10 illustrating one or more diagnostic and/or therapeutic elements, such as tip electrode 26 and ring electrodes 28. It should be understood that the number and arrangement of electrodes 26, 28 is merely illustrative. Indeed, distal region 14 can include any number of electrodes 28, that electrodes 28 may be of various physical configurations (e.g., ring electrodes, segmented ring electrodes, partial ring electrodes) and/or materials (e.g., metallic and/or non-metallic electrodes), that the positioning of electrodes 28 within distal region 14 may vary, and so forth. Moreover, distal segment 14 may include non-electrode diagnostic and/or therapeutic elements, such as positioning sensors, pressure sensors, force sensors, and the like. The term “sensors” will be used herein to refer not only to electrodes 26, 28, but also to other diagnostic and/or therapeutic elements that may be mounted within distal region 14.

One method of manufacture of catheter 10, and in particular of distal region 14 of catheter body 12, according to an embodiment of the present disclosure will be described with reference to FIGS. 3-9. For convenience of explanation, as they are assembled into catheter body 12, the various components described below will be collectively referred to as a “catheter body assembly.”

FIG. 3 illustrates a portion of catheter body 12, and, in particular, distal region 14 thereof. As shown to good advantage in FIG. 3, catheter body 12 generally includes a tubular wall 30 that defines a central lumen 21. A reinforcing layer 32, such as a braided mesh or helically wound reinforcing layer, may run through at least a portion of wall 30 (e.g., terminating within distal region 14). Wall 30 may be made of one or more longitudinally- or radially-arranged segments, joined together such as by reflow bonding, with the various segments made of the same or differing materials depending on the desired characteristics and/or intended use of catheter 10.

Insofar as those of ordinary skill in the art will be generally familiar with the construction of catheter body 12, as may be desirable for a given application of catheter 10, as well as various methods of manufacturing the same, these aspects need not be explained in detail herein. For purposes of illustration, however, the following United States patents and patent application publications, all of which are hereby incorporated by reference as though fully set forth herein, describe suitable structures of and corresponding manufacturing methods for catheter body 12: U.S. Pat. Nos. 8,431,057 ; 8,647,323 ; and U.S. patent application publication no. 2018/0185610. Of course, the foregoing references are merely exemplary, and other structures and manufacturing methods are regarded as within the scope of the instant disclosure.

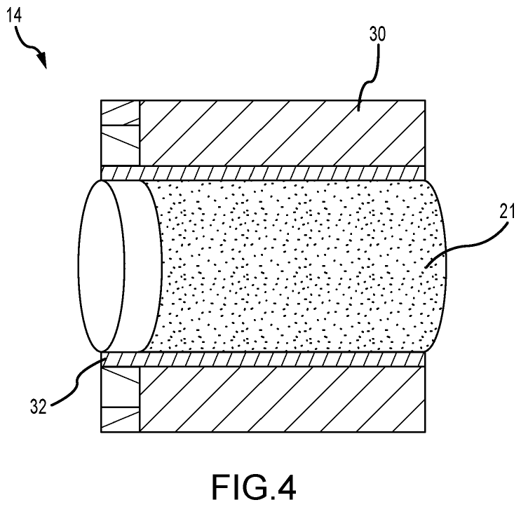

Catheter body 12 may be trimmed to any desired length. For example, FIG. 4 depicts trimming the distal end of catheter body 12 through reinforcing layer 32 (e.g., such that reinforcing layer 32 extends all the way to the distal end of catheter body 12).

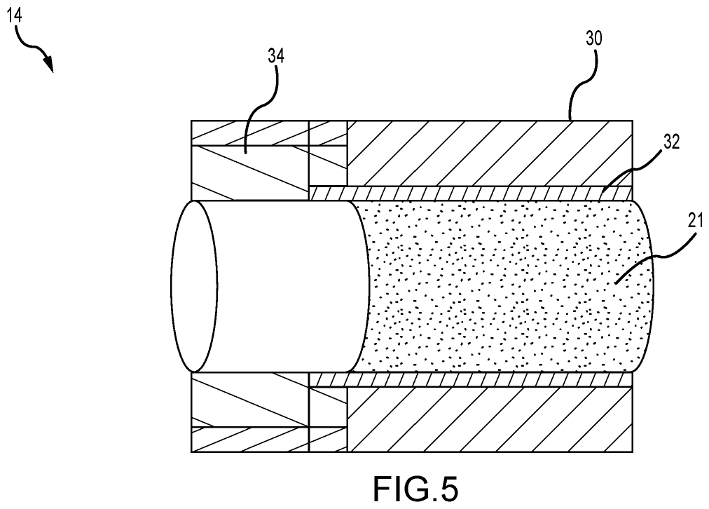

As shown in FIG. 5, a first annular spacing element 34 can be positioned abutting the distal end of catheter body 12. First annular spacing element 34 facilitates accurate positioning of the most proximal sensor (e.g., electrode 28) within distal region 14 (and may thus be trimmed to length as necessary). It also helps prevent shorts between reinforcing layer 32 and this most proximal sensor (e.g., electrode 28). In embodiments of the disclosure, first annular spacing element 34 can be adhesively joined to the distal end of catheter body 12. In other embodiments of the disclosure, first annular spacing element 34 can be reflow bonded to the distal end of catheter body 12.

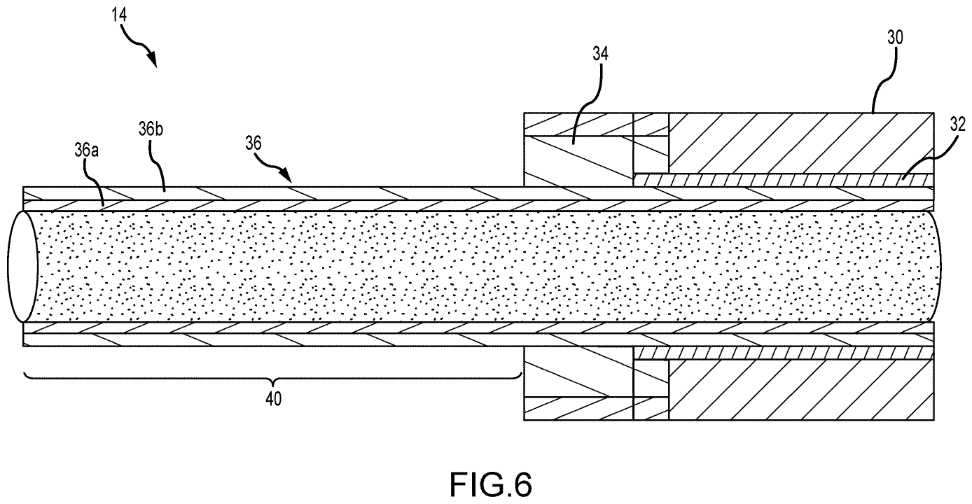

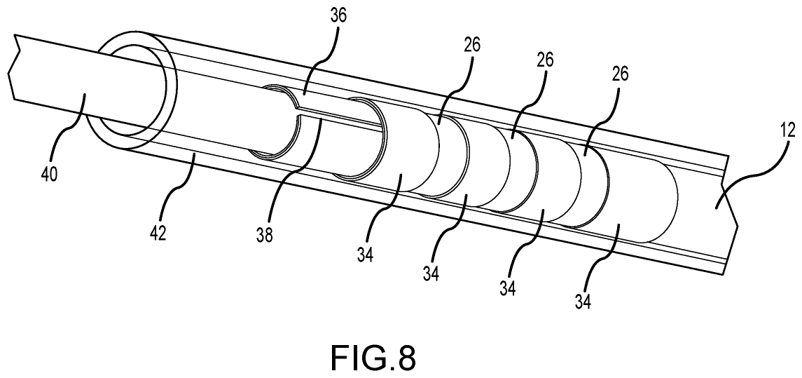

To facilitate construction of a sensor stack abutting the distal end of catheter body 12, FIG. 6 depicts the insertion of a hollow shaft 36 into central lumen 21. A slit 38 (visible in the perspective view of FIG. 8) extends through the wall of hollow shaft 36 from its distal end to its proximal end. Slit 38 allows the diameter of hollow shaft 36 to be temporarily reduced during insertion of hollow shaft 36 into central lumen 21. Distal segment 40 of hollow shaft 36 protrudes beyond the distal end of catheter body 12, thus offering a substrate upon which a sensor stack can be assembled.

Hollow shaft 36 includes an inner layer 36a of a first material and an outer layer 36b of a second material. In embodiments of the disclosure, the first material is a non-reflowable material and the second material is a reflowable material.

In other embodiments of the disclosure, both the first and second materials are reflowable materials, but the melting temperature of the first material is higher than the melting temperature of the second material. Thus, during reflow processing, only outer layer 36b will melt and flow, and inner layer 36a will retain its structural integrity throughout.

In one exemplary embodiment, both inner layer 36a and outer layer 36b are thermoplastic elastomers. For example, inner layer 36a can be Pebax® 72D (Arkema S. A., France), and outer layer 36b can be Pebax® 40D (Arkema S. A., France). Those of ordinary skill in the art will appreciate how to select other suitable materials for hollow shaft 36.

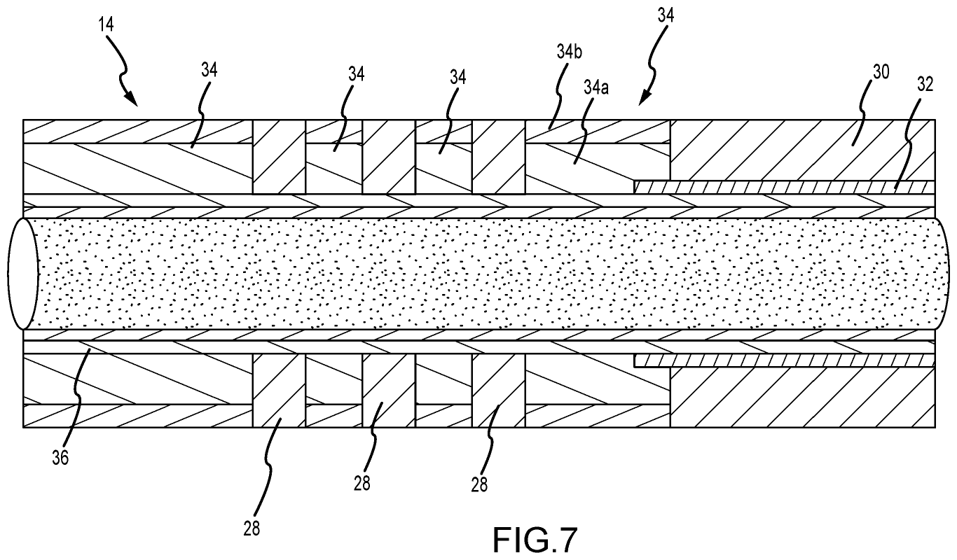

FIG. 7 depicts a sensor stack formed on the protruding distal segment 40 of hollow shaft 36. As shown in FIG. 7, annular spacing elements 34 are alternately disposed with sensors (e.g., electrodes 28). Each annular spacing element 34 facilitates proper axial positioning of the next-most-distal electrode 28 (or other sensor); it also helps prevent shorts between successive electrodes 28 (or other sensors).

FIG. 7 illustrates a total of four annular spacing elements 34 and a total of three electrodes 28. As discussed above, however, the particular number and arrangement of annular spacing elements 34 and electrodes 28 depicted in FIG. 7 is merely illustrative, and that other numbers, arrangements, configurations, and the like are regarded as within the scope of the instant disclosure.

Analogous to inner layer 36a and outer layer 36b of hollow shaft 36, each annular spacing element 34 includes an inner layer 34a of a first material and an outer layer 34b of a second material. For reasons that will become apparent, the layers of annular spacing element 34, however, are the reverse of the layers of hollow shaft 36.

Thus, in embodiments of the disclosure, the first material of inner layer 34a is a reflowable material and the second material of outer layer 34b is a non-reflowable material. Alternatively, both the first and second materials can be reflowable materials, but the melting temperature of the first material can be lower than the melting temperature of the second material. Thus, during reflow processing, only inner layer 34a will melt and flow, and outer layer 34b will retain its structural integrity throughout.

In one exemplary embodiment, both inner layer 34a and outer layer 34b are thermoplastic elastomers. For example, inner layer 34 a can be Pebax® 40D (Arkema S. A., France), and outer layer 34b can be Pebax® 72D (Arkema S. A., France). Those of ordinary skill in the art will appreciate how to select other suitable materials for hollow shaft 34.

Signal conductors 39 that may be coupled to electrodes 28 (or other sensors) can be passed through slit 38 and into central lumen 21, and then routed to proximal end 16 of catheter body 12 for interconnection.

As shown in FIG. 8, a mandrel 40 can be inserted into hollow shaft 36 in order to maintain the patency of central lumen 21 during reflow processing of the catheter body assembly. Mandrel 40 will also press radially outward on hollow shaft 36, pressing the outer diameter of hollow shaft 36 into the inner diameters of annular spacing elements 34 and electrodes 28 (or other sensors).

Similarly, a layer of heat shrink material 42 can be placed around the catheter body assembly. Heath shrink 42 may be a fluoropolymer or polyolefin material such as polytetrafluoroethylene (PTFE) or fluorinated ethylene-propylene copolymer (FEP).

The catheter body assembly may then be reflow processed. Energy (e.g., radiofrequency energy or thermal energy) is applied to the catheter body assembly, for example to the outer surface of the catheter body assembly, in order to heat it to a point above the melting temperatures of inner layer 34a of annular spacing elements 34 and outer layer 36b of hollow shaft 36, but below the melting temperatures of outer layer 34b of annular spacing elements 34 and inner layer 36a of hollow shaft 36. Thus, because of their relative melting temperatures, inner layer 34a of annular spacing elements 34 and outer layer 36b of hollow shaft 36 will melt; outer layer 34b of annular spacing elements 34 and inner layer 36a of hollow shaft 36 will not melt. Heat shrink 42 also has a higher melting or softening temperature such that, during the reflow process, heat shrink 42 will contract while retaining its tubular shape.

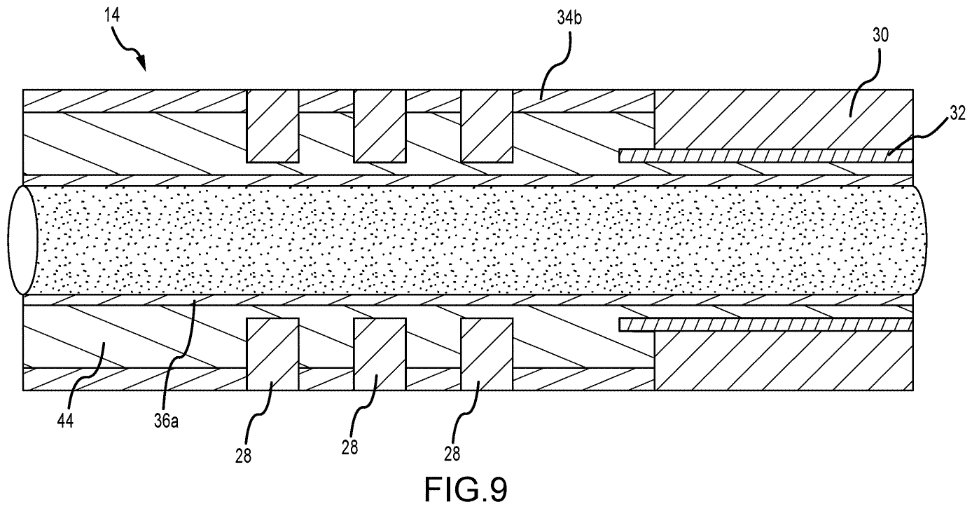

The combination of applied energy and the pressure exerted by heat shrink 42 will force melted inner layer 34a of annular spacing elements 34 and melted outer layer 36b of hollow shaft 36 to flow and redistribute about the catheter body assembly. Once cooled, they will bond together into a substantially continuous layer 44, sandwiched between outer layer 34b of annular spacing elements 34 and inner layer 36a of hollow shaft 36. This arrangement is shown in FIG. 9, which further illustrates that layer 44 offers additional sealing against fluid ingress around electrodes 28.

It should also be noted that the inclusion of slit 38 in hollow shaft 36 will leave a witness line visible through annular spacing elements 34.

Once the assembly has cooled, mandrel 40 can be removed, leaving central lumen 21. Heat shrink 42 may also be removed, if desired.

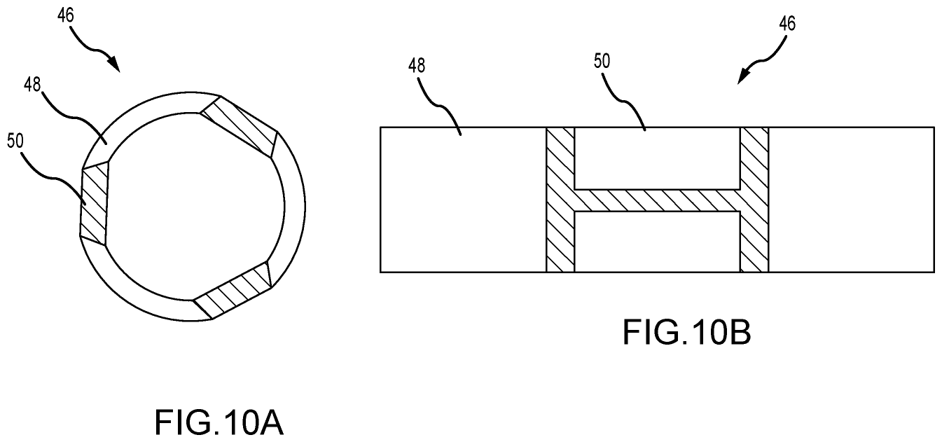

According to aspects of the disclosure, the sensors can be a plurality of composite ring sensors 46 as depicted in FIGS. 10A and 10B. Ring electrodes 46 include a non-conductive annular body 48 that defines a plurality of cavities into which a respective plurality of conductive elements 50 can be disposed.

Although several embodiments have been described above with a certain degree of particularity, those skilled in the art could make numerous alterations to the disclosed embodiments without departing from the spirit or scope of this invention.

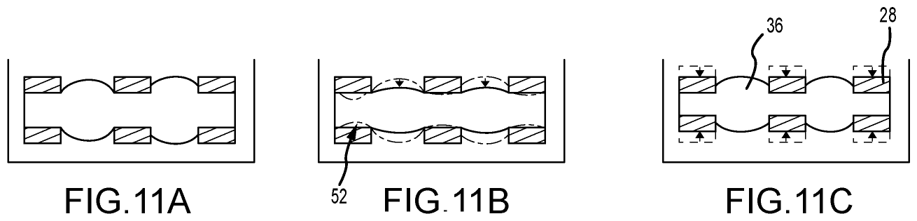

For example, in embodiments of the disclosure, a swaging process may be used in addition to, or as an alternative to, the sensor mounting process described above. For example, as shown in FIG. 11A, electrodes 28 (or other sensors) may be initially secured to hollow shaft 36 (or another suitable tubular substrate) via a primary swaging step. Subsequent processing (e.g., the reflow processing described above) may result in dimensional changes (e.g., heat-induced stressed relaxation of shaft 36) that alter the spacing 52 between electrodes 28 and shaft 36, as shown in FIG. 11B. To further reinforce the connection between electrodes 28 and shaft 36, therefore, a secondary swaging step can be performed as shown in FIG. 11C, thus reducing the diameter of electrodes 28 by about an additional 0.0001 inches.

All directional references (e.g., upper, lower, upward, downward, left, right, leftward, rightward, top, bottom, above, below, vertical, horizontal, clockwise, and counterclockwise) are only used for identification purposes to aid the reader's understanding of the present invention, and do not create limitations, particularly as to the position, orientation, or use of the invention. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other.

It is intended that all matter contained in the above description or shown in the accompanying drawings shall be interpreted as illustrative only and not limiting. Changes in detail or structure may be made without departing from the spirit of the invention as defined in the appended claims.

1. A method of securing at least one sensor to a medical device, comprising:
providing a tubular medical device body defining a central lumen;
inserting a hollow shaft into the central lumen, wherein the hollow shaft comprises:
an inner layer of a first material;
an outer layer of a second material,
wherein a melting temperature of the second material is lower than a melting temperature of the first material; and
a slit through the inner layer and the outer layer and extending from a proximal end of the hollow shaft to a distal end of the hollow shaft,
wherein a distal segment of the hollow shaft protrudes beyond a distal end of the tubular medical device body;
forming a sensor stack around the protruding distal segment of the hollow shaft and abutting the distal end of the tubular medical device body, the sensor stack comprising:
at least one annular spacing element comprising an inner layer of a third material and an outer layer of a fourth material, wherein a melting temperature of the third material is lower than a melting temperature of the fourth material; and
at least one annular sensor; and
reflow bonding the sensor stack and the tubular medical device body to the hollow shaft.

2-4. (canceled)

5. The method according to claim 1, wherein the at least one annular spacing element comprises a plurality of annular spacing elements alternately arranged with the at least one annular sensor.

6. The method according to claim 5, wherein the at least one annular sensor comprises a plurality of annular sensors alternately arranged with the plurality of annular spacing elements.

7. The method according to claim 1, wherein the at least one annular spacing element abuts the distal end of the tubular medical device body.

8. The method according to claim 7, further comprising securing the at least one annular spacing element to the distal end of the tubular medical device body via an adhesive.

9. The method according to claim 1, wherein the at least one annular sensor comprises at least one ring electrode.

10. The method according to claim 9, wherein the at least one ring electrode comprises at least one composite ring sensor comprising:
a non-conductive annular body defining a plurality of cavities; and
a plurality of conductive elements respectively disposed in the plurality of cavities.

11. (canceled)

12. The method according to claim 1, further comprising:
coupling at least one signal conductor respectively to the at least one annular sensor; and
routing the at least one signal conductor through the slit of the hollow shaft and into the central lumen of the tubular medical device body.

13. (canceled)

14. The method according to claim 1, wherein reflow bonding the sensor stack and the tubular medical device body to the hollow shaft comprises heating the sensor stack and the tubular medical device to a temperature above the melting temperature of the second material and the melting temperature of the third material but below the melting temperature of the first material and the melting temperature of the fourth material.

15. The method according to claim 1, further comprising:
swaging the at least one annular sensor onto the protruding distal segment of the hollow shaft prior to the reflow bonding step in a primary swaging step; and
swaging the at least one annular sensor onto the protruding distal segment of the hollow shaft after the reflow bonding step in a secondary swaging step.

16. (canceled)

17. A medical device, comprising:
a tubular medical device body defining a central lumen;
a hollow shaft inserted within the central lumen, wherein the hollow shaft comprises:
an inner layer of a first material;
an outer layer of a second material,
wherein a melting temperature of the second material is lower than a melting temperature of the first material; and
a slit through the inner layer and the outer layer and extending from a proximal end of the hollow shaft to a distal end of the hollow shaft,
wherein a distal segment of the hollow shaft protrudes beyond a distal end of the tubular medical device body; and
a sensor stack around the protruding distal segment of the hollow shaft and abutting the distal end of the tubular medical device body, wherein the sensor stack comprises:
at least one annular spacing element comprising an inner layer of a third material and an outer layer of a fourth material, wherein a melting temperature of the third material is lower than a melting temperature of the fourth material; and
at least one annular sensor; and
wherein the sensor stack and the tubular medical device body are reflow bonded to the hollow shaft.

18. The medical device according to claim 15, wherein the at least one annular spacing element comprises a plurality of annular spacing elements alternately arranged with the at least one annular sensor.

19. The medical device according to claim 16, wherein the at least one annular sensor comprises a plurality of annular sensors alternately arranged with the plurality of annular spacing elements.

20. The medical device according to claim 15, wherein the at least one annular spacing element abuts the distal end of the tubular medical device body.

21. The medical device according to claim 18, wherein the at least one annular spacing element is adhesively secured to the distal end of the tubular medical device body.

22. The medical device according to claim 15, wherein the at least one annular sensor comprises at least one ring electrode.

23. The medical device according to claim 20, wherein the at least one ring electrode comprises at least one composite ring sensor.

24. The medical device according to claim 21, wherein the at least one composite ring sensor comprises:
a non-conductive annular body defining a plurality of cavities; and
a plurality of conductive elements respectively disposed in the plurality of cavities.

25. The medical device according to claim 15, further comprising at least one signal conductor respectively coupled to the at least one annular sensor and routed through the slit of the hollow shaft and into the central lumen of the tubular medical device body.

26. A method of manufacturing a medical device, comprising:
providing a tubular medical device body defining a central lumen;
inserting a hollow shaft into the central lumen, wherein the hollow shaft comprises an inner layer of a non-reflowable material, an outer layer of a reflowable material, and a slit through the inner layer and the outer layer and extending from a proximal end of the hollow shaft to a distal end of the hollow shaft, such that a distal segment of the hollow shaft protrudes beyond a distal end of the tubular medical device body;
forming a sensor stack around the protruding distal segment of the hollow shaft and abutting the distal end of the tubular medical device body, wherein the sensor stack comprises a plurality of annular spacing elements alternately arranged with a plurality of annular sensors, and wherein each annular spacing element comprises an inner layer of a reflowable material and an outer layer of a non-reflowable material;
inserting a mandrel into the hollow shaft; and
reflow bonding the respective inner layers of the plurality of annular spacing elements to the outer layer of the hollow shaft.
